(19) United States
(12) Patent Application Publication
(10) Pub. No.: US 2026/0053887 A1
(43) Pub. Date: Feb. 26, 2026

(54) Methods and Kits to Prevent And/Or Treat Cardiac Disorders

(71) Applicant: UNIVERSITY OF IOWA RESEARCH FOUNDATION, Iowa City, IA (US)

(72) Inventor: Ajit Vikram, Iowa City, IA (US)

(21) Appl. No.: 19/306,273

(22) Filed: Aug. 21, 2025

Related U.S. Application Data
(60) Provisional application No. 63/685,433, filed on Aug. 21, 2024.

(51) Int. Cl. A61K 38/14; A61K 31/198; A61K 31/404; A61K 31/4045; A61K 31/405; A61K 31/44; A61K 31/47; A61P 9/04

(52) U.S. Cl. CPC A61K 38/14; A61K 31/198; A61K 31/404; A61K 31/4045; A61K 31/405; A61K 31/44; A61K 31/47; A61P 9/04

(57) ABSTRACT

Methods and kits for treating and/or preventing a cardiac disorder are disclosed. The methods can include administering an indole metabolite to a subject. The methods can also include administering a glycopeptide antibiotic to the subject.

### CROSS-REFERENCE TO RELATED APPLICATIONS

This application claims the benefit of U.S. Provisional Application Ser. No. 63/685,433, filed Aug. 21, 2024, the entire content of which is incorporated by reference in its entirety.

### STATEMENT REGARDING FEDERALLY SPONSORED RESEARCH

This invention was made with government support under R01HL16777301A1, awarded by the National Institutes of Health. The government has certain rights in the invention.

### BACKGROUND

Antibiotic use decreases intestinal microbial diversity and is associated with an increased risk of heart failure. Antibiotics alter the composition of the intestinal microbiome, leading to the expansion of pathogenic microbes, which are associated with an increased risk of cardiovascular events. The cardiovascular health benefits of prebiotics, probiotics, and bariatric surgery implicate microbiota's role. Conversely, a diet rich in fiber and bacteria-derived short-chain fatty acids to restore gut health protects against myocardial hypertrophy, whereas a diet rich in microbiota-derived trimethylamine N-oxide promotes myocardial hypertrophy. Further, the antibiotics-induced disruption of intestinal microbiota exacerbates the pressure overload-induced cardiac hypertrophy, and microbiota-derived peptide mimics drive lethal inflammatory cardiomyopathy.

### SUMMARY

In one aspect, a method for treating and/or preventing a cardiac disorder is provided. The method can include administering one or more indole metabolites to a subject in need thereof.

In another aspect, a kit for treating and/or preventing a cardiac disorder is provided. The kit can include one or more indole metabolites and a glycopeptide antibiotic.

### BRIEF DESCRIPTION OF THE DRAWINGS

FIGS. 1A-1M. Vancomycin inhibits TAC-induced heart failure by reshaping the gut microbiota. (FIGS. 1A-1C) Effect of vancomycin, ampicillin, and neomycin on heart weight (HW) normalized to tibia length (TL) (FIG. 1A), peak longitudinal strain (FIG. 1B), and representative speckle tracing images (FIG. 1C) of heart at systole and diastole five weeks after TAC surgery. n=7-12. The mice were given antibiotics daily beginning one week before TAC surgery, and data was collected five weeks after TAC surgery. (FIG. 1D) Images showing the fibrosis (Sirius red) and cross-sectional area of cardiomyocytes (wheat-germ agglutinin). (FIG. 1E) Effect of vancomycin, ampicillin, and neomycin on the expression of load-response genes (nppa, nppb, β-mhc) in the heart of mice five weeks after TAC surgery. n=7-12. (FIG. 1F) Principal component analysis of intestinal microbiota shows the relative distance of each experimental group. n=3-5. (FIG. 1G) Heatmap showing Pearson correlation-based hierarchical clustering of ASVs (16S rRNA sequencing, rows) and experimental groups (columns). The enlarged panel at the bottom shows select amplicon sequence variants (ASVs) in the TAC-VF group that exhibits a similar pattern as that of TAC-V mice. (FIGS. 1H-1K) The mice receiving fecal microbiota from vancomycin-treated mice (TAC-VF) had a smaller heart (FIG. 1H), lower cardiac strain (FIGS. 1I and 1J), lower fibrosis (FIG. 1K), and smaller cross-sectional area (FIG. 1K) than mice that received microbiota from saline-treated mice (TAC-SF) following TAC (5 weeks). n=5. The dotted line in FIG. 1H shows the HW/TL of sham mice for comparison. Images in FIG. 1J show the speckle tracking-based cardiac longitudinal strain at systole and diastole. (FIGS. 1L and 1M) Echocardiography-based LV wall thickness of mice that received saline or vancomycin for three weeks beginning 2 weeks after TAC. (FIG. 1M) Vancomycin for three weeks beginning 2 weeks after TAC inhibits myocardial hypertrophy (TAC-2V). Heart images are captured five weeks after sham or TAC surgery. n=5. * p<0.05, **p<0.01, ***p<0.001, vs. indicated group. Data: mean±s.e.m. Sham; control, TAC; mice underwent TAC, TAC-V/A/N; TAC mice receiving vancomycin/ampicillin/neomycin.

FIGS. 2A-2M. Vancomycin reshapes gut microbiota and increases tryptophan catabolism to tryptamine to mediate cardioprotection. (FIG. 2A) PCA showing that vancomycin induces a distinct change in fecal metabolites during TAC. n=6. (FIG. 2B) Heatmap showing Pearson correlation-based hierarchical clustering of fecal metabolites in Sham, TAC, and TAC-V. The enlarged panel at the bottom shows tryptophan's microbial and host (mammalian) metabolites. n=6. Figure insert shows the relative tryptamine levels (arbitrary unit). n=6. (FIG. 2C) PCA showing that vancomycin induces a distinct change in the serum metabolites during TAC. n=6. (FIG. 2D) Heatmap showing hierarchical clustering of serum metabolites in Sham, TAC, and TAC-V. The enlarged panel at the bottom shows levels of tryptophan metabolites. n=6. Figure insert shows the relative tryptamine levels. Note that the arbitrary unit is the same as in feces, suggesting a higher tryptamine in the serum. n=6. (FIG. 2E) Integrative metabolic model-based microbiome and metabolomics data analysis showing that Lactobacillus sp. is strongly associated with tryptophan variance. (FIG. 2F) Mice that received tryptamine (12.5 mg/kg, TAC-TM) or indole aldehyde (12.5 mg/kg/day), but not tryptophan (500 mg/kg, TAC-TY), are protected from TAC-induced myocardial hypertrophy. n=6-8. The dotted line shows WT-Sham mice's heart weight to tibia length (HW/TL). (FIGS. 2G-2I) The oral supplementation of tryptamine increases the serum levels of tryptamine while tryptophan does not as evident from the heartmap (FIG. 2G) and quantification of serum tryptamine and tryptophan levels (FIGS. 2H and 2I). (FIGS. 2J and 2K) Mice that received tryptamine (12.5 mg/kg, TAC-TM), but not tryptophan (500 mg/kg, TAC-TY), are protected from TAC-induced strain (FIG. 2J), fibrosis (FIG. 2K), and cardiomyocyte enlargement (FIG. 2K). n=6-8. (FIGS. 2L and 2M) The mice receiving vancomycin two weeks after TAC surgery also had higher levels of tryptamine in the feces, as evidenced by the heatmap and quantification. n=4-6. *p<0.05, **p<0.01, ***p<0.001, vs. indicated group. Data: mean±s.e.m.

FIGS. 3A-3I. Vancomycin-induced reshaping of gut microbiota improves mitochondrial function. (FIG. 3A) Schematic summarizing the 02K assay. (FIG. 3B) Effect of pyruvate/malate (mimicking basal condition) and ADP (mimicking stressed condition) on the oxygen consumption by the permeabilized cardiac fibers of the Sham, TAC, and TAC-V mice. n=12-24 (3-5 mice). (FIGS. 3C and 3D) The expression of the mitochondrial electron transport chain complexes CI-CV and its quantification. (FIG. 3E) Transmission electron microscopy of the heart of Sham, TAC, and TAC-V mice. Magnification ×1,000, ×7,000, and ×20,000. Please note the shape and membrane prominence of the mitochondria in the heart of Sham and TAC-V mice compared to that of the TAC mice. FIG. 3F-3I) Immunoblots show the expression of mitophagy-related proteins and their quantification in total heart lysate (FIGS. 3F and 3G) and the mitochondrial fraction (FIGS. 3H and 3I) of the heart of Sham, TAC, and TAC-V mice. *p<0.05, **p<0.01, ***p<0.001, vs. indicated group. Data: mean±s.e.m. Statistics: One-way ANOVA.

### DETAILED DESCRIPTION

Aspects of the present disclosure relate, in part, to methods and kits for treating and/or preventing a cardiac disorder in a subject. In various aspects, the methods can include administering one or more indole metabolite to a subject. In one or more aspects, the indole metabolite can include one or more of tryptophan, tryptamine, indole propionic acid, indole aldehyde, indole acrylic acid, tryptophol, kynurenine, quinolinic acid, and xanthurenic acid. In certain aspects, the indole metabolite can be tryptamine or indole aldehyde. The structure of tryptamine and indole aldehyde are provided in Formula I and II below, respectively:

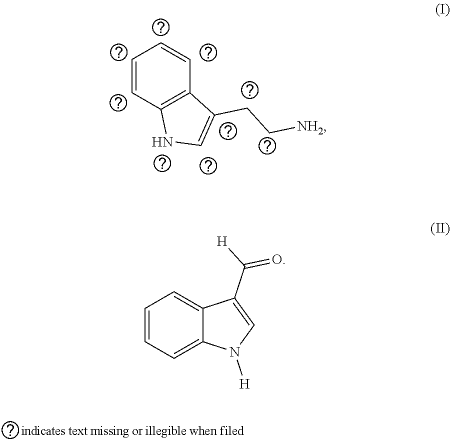 [SMILES: NCCc1c[nH]c2ccccc12 ; O=Cc1c[nH]c2ccccc12]

In various aspects, the indole metabolite can be any metabolite of an indole-containing compound, e.g., that is metabolized, as least partly, by bacteria and/or one or more microbial species found in the gut of a subject, for example a human or other mammal. In one or more aspects, the indole metabolite can be a metabolite of an indole-containing compound that is metabolized, at least partly, by Lactobacillus sp.

### Methods

In one aspect of this disclosure, a method for treating and/or preventing a cardiac disorder is provided that includes administering one or more indole metabolites to a subject in need thereof. In various aspects, the one or more indole metabolite can be administered to the subject in an amount of about 10 milligrams (mg)/kilogram (kg)/day to about 500 mg/kg/day, or about 25 mg/kg/day to about 250 mg/kg/day.

In various aspects, the indole metabolite can be administered to the subject according to any regimen suitable for cardio protection and/or treatment. In certain aspects, the indole metabolite can be administered daily, weekly, or monthly.

In various aspects, the indole metabolite can be present in a comestible product or composition and/or in a nutritional supplement. The indole metabolite can be present in a comestible product, composition, nutritional supplement, or combinations thereof. Any suitable comestible products or compositions and/or nutritional supplements can be utilized.

In one or more aspects, the indole metabolite can be administered orally, subcutaneously, intravenously, transdermal patches, or any other suitable route of administration.

In one or more aspects, the methods can include administering one or more glycopeptide antibiotic to the subject. In various aspects, the glycopeptide antibiotic can be administered to the subject prior to administering the indole metabolite.

In certain aspects, the glycopeptide antibiotic can include one or more of vancomycin, teicoplanin, ramoplanin, oritavancin, dalbavancin, and telavancin. In one aspect, the glycopeptide antibiotic can include vancomycin.

In various aspects, the glycopeptide antibiotic can be administered to the subject from about 10 mg/kg/day to about 2000 mg/kg/day.

In one or more aspects, the glycopeptide antibiotic can be administered to the subject according to any regimen suitable for cardio protection and/or treatment. In certain aspects, the glycopeptide can be administered daily, weekly, or monthly.

In various aspects, all or part of the glycopeptide antibiotic dosing regimen may be completed prior to commencement of the administration of the indole metabolite. For instance, in one aspect, a subject may take the glycopeptide antibiotic for at least one day, at least two days, at least three days, at least five days, at least seven days, or at least 10 days, prior to indole metabolite administration. In alternative aspects, the glycopeptide antibiotic and the indole metabolite can be taken concurrently and/or with overlapping dosing schedules or regimens.

In one or more aspects, the glycopeptide antibiotic is administered orally or intravenously. In one aspect, the glycopeptide antibiotic is administered orally.

In various aspects, administering the glycopeptide antibiotic can increase the abundance of Lactobacillus sp. in the subject's gut, relative to the subject's gut prior to administering the glycopeptide antibiotic, including relative to the level or amount of Lactobacillus sp. in the gut of the subject prior to administering the glycopeptide antibiotic. In the same or alternative aspects, the glycopeptide antibiotic can decrease the abundance of Bacteroidates, and/or increase the abundance of Firmicutes and/or Proteobacteria in the subject's gut, relative to the subject's gut prior to administering the glycopeptide antibiotic. In aspects, the glycopeptide antibiotic can decrease the abundance of Bacteroidetes in the gut of a subject relative to the level or amount of Bacteroidetes in the gut of the subject prior to administering the glycopeptide antibiotic. In aspects, the glycopeptide antibiotic can increase the abundance of Firmicutes in the gut of a subject relative to the level or amount of Firmicutes in the gut of the subject prior to administering the glycopeptide antibiotic. In aspects, the glycopeptide antibiotic can increase the abundance of Proteobacteria in the gut of a subject relative to the level or amount of Proteobacteria in the gut of the subject prior to administering the glycopeptide antibiotic.

In certain aspects, the subject in need thereof can be one who has, or is at risk of having, one or more cardiac disorders. In certain aspects, the subject in need thereof can exhibit one or more risk factors for heart failure comprising hypertension, obesity, and type 2 diabetes. In various aspects, the subject in need thereof has, or is at risk of, one or more cardiac disorders selected from the group consisting of: heart failure, coronary artery disease, cardiomyopathy, heart valve disease, aortic stenosis, and pericardial disease.

As used in this disclosure, the term “subject” or “individual” or “patient” may be used interchangeably and refer to all animals, including mammals, e.g., a human or non-human, who are prone to or suffering from an indicated disease or disorder, such as one or more cardiac disorders. In certain aspects, the subject can be any mammal. In various aspects, the subject is a human.

In various aspects, methods for treating and/or preventing a cardiac disorder are provided that include administering or delivering a Lactobacillus sp. to a subject in need thereof. For instance, in one aspect, such a method can include administering or delivering a glycopeptide antibiotic to a subject in need thereof. The glycopeptide antibiotic and regimens are discussed in this disclosure. In various aspects, the methods can include administering a Lactobacillus sp. to a subject. In such aspects, the Lactobacillus sp. can be administered to the subject subsequent to, or concurrent with, the administration of the glycopeptide antibiotic.

### Kits

Various aspects of the present disclosure relate to kits for treating and/or preventing one or more cardiac disorders. In one or more aspects, the kits can include one or more indole metabolite and one or more glycopeptide antibiotic as described in this disclosure. In one or more aspects, the indole metabolite can be one or more of tryptophan, tryptamine, indole propionic acid, indole aldehyde, indole acrylic acid, tryptophol, kynurenine, quinolinic acid, and xanthurenic acid. In aspects, the indole metabolite can be tryptamine. In aspects, the indole metabolite can be indole aldehyde. The indole metabolite can be present in a comestible product, or composition, or in a nutritional supplement, or combinations thereof. In various aspects, the glycopeptide antibiotic can include one or more of vancomycin, teicoplanin, ramoplanin, oritavancin, dalbavancin, and/or telavancin. In one aspect, the glycopeptide antibiotic is vancomycin.

In one or more aspects, the kits can include one or more glycopeptide antibiotic and Lactobacillus sp.

The definitions and terminology used herein are for the purpose of describing particular aspects only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, 5, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Aspects of the present disclosure that are described with respect to methods can be utilized in the context of the compositions of matter or kits discussed in this disclosure. Similarly, aspects of the present disclosure that are described with respect to compositions of matter can be utilized in the context of the methods and kits, and aspects of the present disclosure that are described with respect to kits can be utilized in the context of the methods and compositions of matter.

The above description, attached figures, and claims listed below are intended to be illustrative and not limiting of this invention. In light of the invention described herein, many themes and variations to this invention will be suggested to one skilled in the art. All such themes and variations are within the contemplation hereof. For instance, while this invention has been described in conjunction with the various exemplary embodiments outlined above and in the below claims, various alternatives, modifications, variations, improvements, and/or substantial equivalents, whether known or that are rare or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments.

It is to be understood that the invention is not limited to the particular embodiments described. It is also to be understood that the terminology used herein is for the purpose of describing particular embodiments only, and is not intended to be limiting. The scope of the present invention will be limited only by the claims.

It should be apparent to those skilled in the art that many additional modifications beside those already described are possible without departing from the inventive concepts. In interpreting this disclosure, all terms should be interpreted in the broadest possible manner consistent with the context.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

### EXAMPLES

### Example 1—Preventing and Inhibiting Heart Failure Involving Microbial Reshaping and Microbial Metabolite

Antibiotic use decreases intestinal microbial diversity and is associated with an increased risk of heart failure.^{1, 2} Antibiotics alter the composition of the intestinal microbiome³⁻⁵, leading to the expansion of pathogenic microbes,^{6, 7} which are associated with an increased risk of cardiovascular events.^{1, 2, 8-11} The cardiovascular health benefits of prebiotics, probiotics, and bariatric surgery implicate microbiota's role.¹²⁻²⁷ Conversely, a diet rich in fiber and bacteria-derived short-chain fatty acids to restore gut health protects against myocardial hypertrophy,⁹ whereas a diet rich in microbiota-derived trimethylamine N-oxide promotes myocardial hypertrophy.¹⁰ Further, the antibiotics-induced disruption of intestinal microbiota exacerbates the pressure overload-induced cardiac hypertrophy,²⁸ and microbiota-derived peptide mimics drive lethal inflammatory cardiomyopathy.²⁹ These studies highlight the critical connection between the intestinal microbiota and the heart, show that the disturbed intestinal microbiota increases the risk of heart failure, and microbes that confer resistance to it are present under homeostatic conditions. However, a distinct change in the microbiota by a pharmacological agent that improves cardiac resilience to hypertrophic stress is counterintuitive and remains unknown. Vancomycin induces a distinct intestinal microbiota profile characterized by an increase in some genus/species of the phyla Firmicutes (e.g., Lactobacillus sp.),³⁰⁻³² that improves cardiac health.³³ Herein, a method that uses antibiotics-induced microbial reshaping, which changes the microbial generation of indole metabolites and leads to cardiac disorder (e.g. heart failure) mitigation is described.

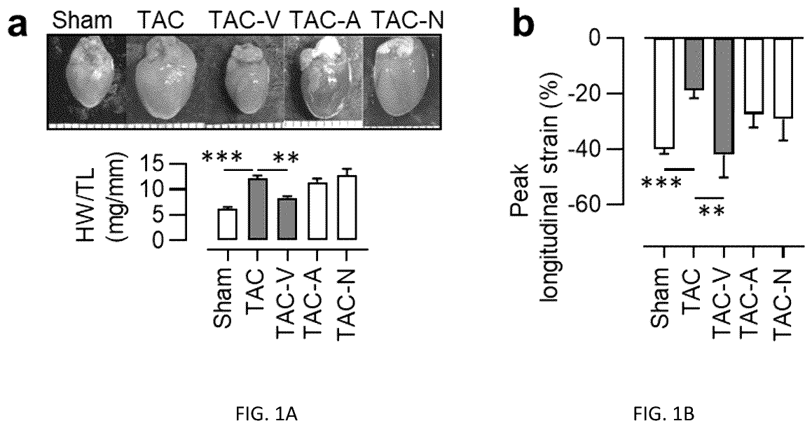

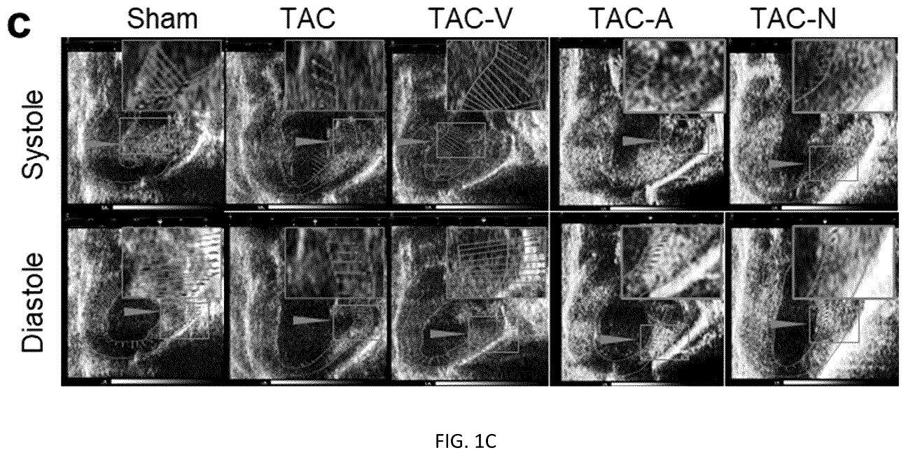

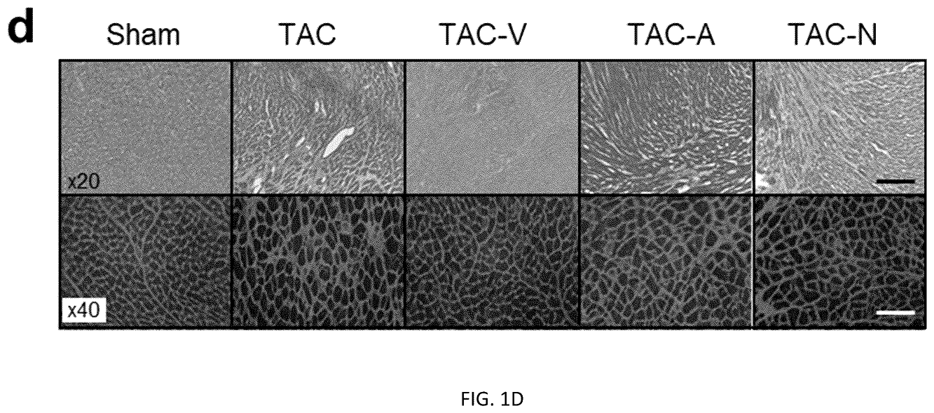

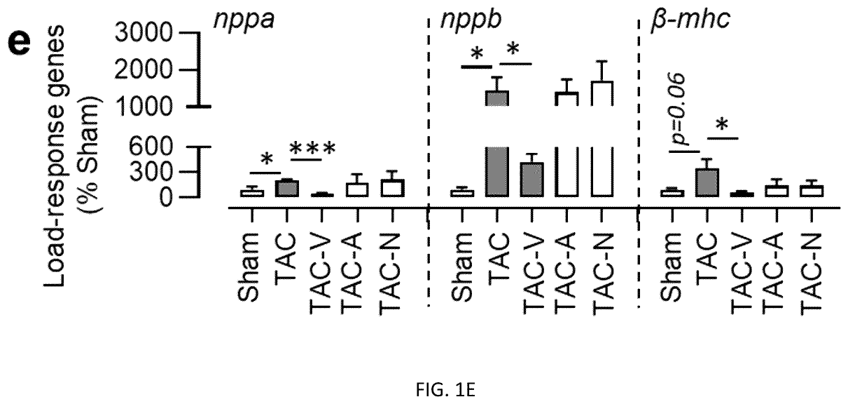

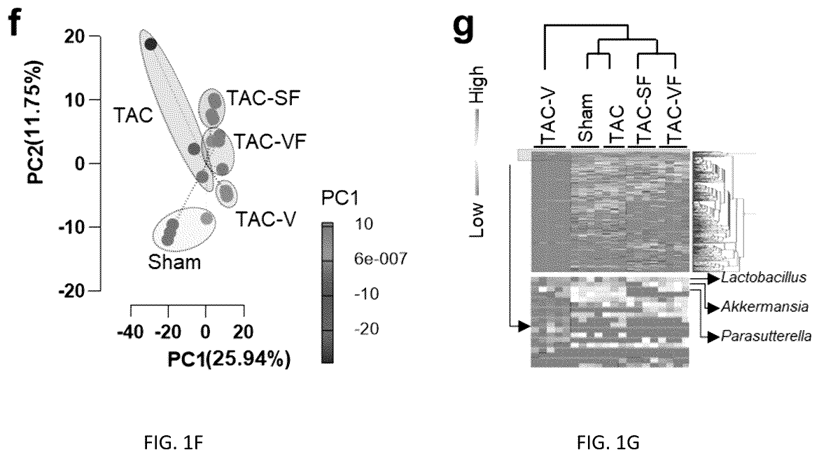

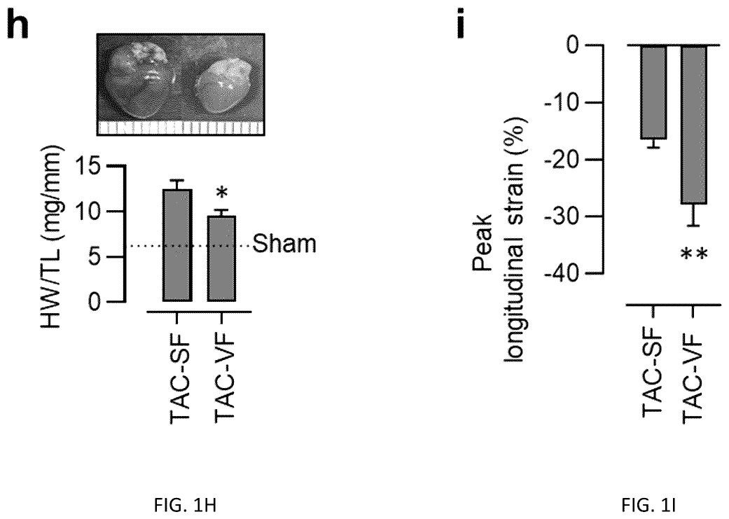

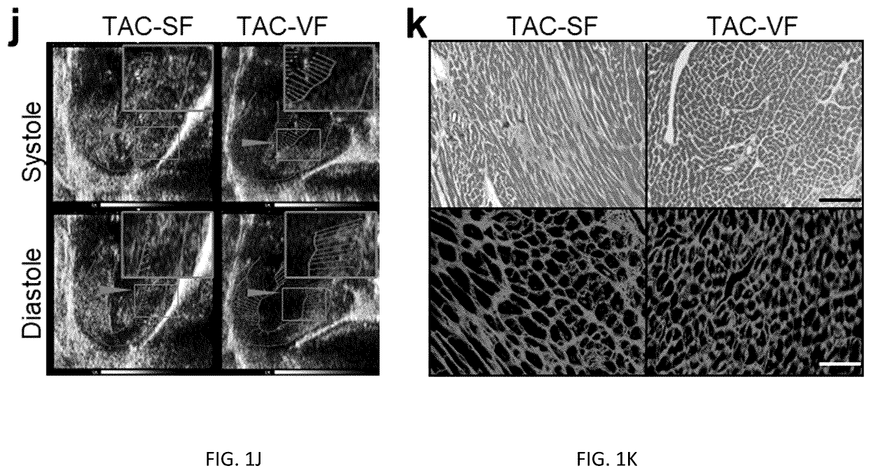

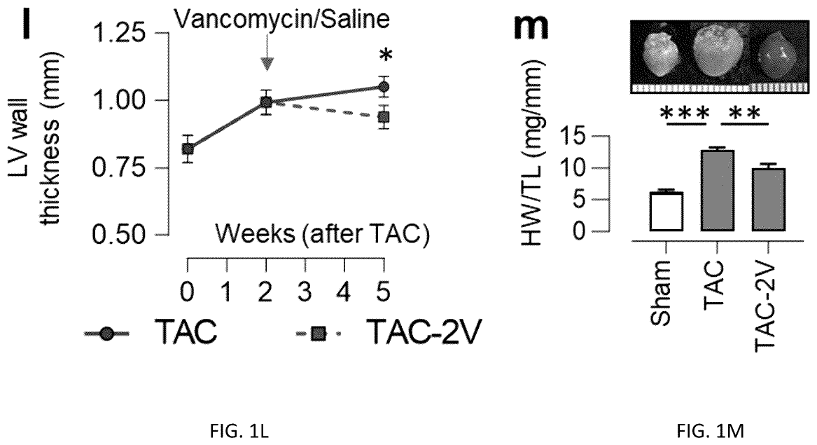

Vancomycin reshapes microbiota in the gut and inhibits pressure overload-induced myocardial hypertrophy and fibrosis. To determine the effects of vancomycin on myocardial hypertrophy, the vancomycin was provided orally one week before the Trans-aortic constriction (TAC) surgery and continued until five weeks after the TAC surgery, and functional and morphometric cardiac parameters were assessed. We find that the vancomycin-treated TAC mice (TAC-V) did not develop myocardial hypertrophy and had a higher percent ejection fraction (% EF) than saline-treated TAC mice (FIG. 1A). Two-dimensional speckle tracking echocardiography-derived myocardial strain is a sensitive marker of left ventricular dysfunction.³⁴ We found a significant decrease in the peak and global longitudinal strain, left ventricular weight and wall thickness, and end-systolic/diastolic volume in TAC-V mice compared to the TAC mice (FIGS. 1B and 1C). A lower occurrence of fibrosis and lower cross-sectional area of cardiomyocytes in the hearts of TAC-V mice than that of TAC mice was found (FIG. 1D). Moreover, the hearts of TAC-V mice had a decreased expression of load-response genes (nppa, nppb, and β-mhc) and collagen genes (Col1a1, Col1a2, and Col3a1), supporting that the mechanisms that contribute to fibrosis were not activated in the heart of TAC-V mice (FIG. 1E). In contrast, microbial reshaping using ampicillin (TAC-A) and neomycin (TAC-N) did not protect the TAC-induced myocardial hypertrophy and dysfunction (FIGS. 1A-IE), suggesting that the cardioprotective effects are specific to vancomycin. As antibiotics affect intestinal microbial diversity, whether vancomycin-induced microbial reshaping is responsible for its cardioprotective effects was investigated. The principal component analysis (PCA), signifying β-diversity, of fecal microbiota profile (16S rRNA) shows that the TAC-V mice have a distinct microbiota profile than Sham and TAC mice (FIG. 1F). The vancomycin decreases the abundance of Bacteroidates, while increases that of Firmicutes and Proteobacteria in humans and mice,³² and we found a similar global effect of vancomycin in mice on the abundance of Bacteroidates, Firmicutes, and Proteobacteria. A set of microbes minimally present in the gut of Sham and TAC mice (e.g., Lactobacillus, Akkermansia, Parasutterella) had strikingly higher levels in the TAC-V mice (FIG. 1G). The similarity index (Pearson correlation) also shows a closeness between sham and TAC mice than TAC-V mice. Next, we asked whether the transfer of microbiota from the vancomycin-treated donor mice to the TAC mice can transfer the cardioprotective effects of vancomycin. The microbiota-depleted mice (by using a cocktail of broad-spectrum antibiotics for one week^{35, 36}) were gavaged with the fecal microbiota of vancomycin/saline-treated donor mice every day beginning one day after the TAC surgery and were continued until five weeks after TAC surgery. Vancomycin is poorly absorbed through the intestinal epithelium, and we did not notice any difference in the blood levels of FITC dextran after its oral administration in the control and vancomycin-treated mice, suggesting that the vancomycin does not change intestinal permeability. Therefore, the fecal content of vancomycin-treated mice is likely to contain vancomycin. However, the presence of vancomycin in fecal pellets does not pose a problem since dilution during the microbiota transfer protocol substantially (˜100-fold) reduces the overall amount of vancomycin received by the recipient animals. In addition, the microbes that survived in the feces of vancomycin-treated mice would also be resistant to the residual vancomycin. The fecal microbiota profile of TAC mice that received microbiota from the vancomycin-treated donor mice (TAC-VF) was closer to TAC-V mice than that of the mice that received microbiota from the saline-treated mice (TAC-SF) (FIGS. 1F and 1G). We found that the TAC-VF mice developed milder myocardial hypertrophy and cardiac strain than the TAC-SF mice (FIG. 1H-1J). In addition, a significant decrease in the left ventricular weight/wall thickness, improved % EF, and end-systolic/diastolic volume were observed in the TAC-VF mice than in TAC-SF mice, indicating lower susceptibility to myocardial hypertrophy and dysfunction. We also found decreased fibrosis, load-response genes (nppb, nppa, and β-mhc), collagen genes (Col1a1, Col1a2, and Col3a1), and cross-sectional area of cardiomyocytes in the hearts of TAC-VF mice (FIG. 1K), like our observations in the TAC-V mice. We did not notice any significant change in the heart rate of TAC-VF mice compared to TAC-SF mice. The vancomycin mainly increased the intestinal abundance of amplicon sequence variants (ASV) 0001 and 0003, and by reference sequence query in the blastn database, it best resembled Lactobacillus murinus and Lactobacillus animalis.

Vancomycin reshapes microbiota in the gut and treats pressure overload-induced myocardial hypertrophy and fibrosis. We asked if vancomycin could reverse the early hypertrophic changes in the heart. TAC submaximally induces cardiac hypertrophy in the first two weeks.³⁷ The echocardiographic assessment shows that vancomycin treatment beginning two weeks after TAC surgery and continued for the next three weeks (TAC-2V) reversed TAC-induced LV wall thickness and weight (FIG. 1L). The heart weight of TAC-2V mice was also significantly less than that of the TAC mice (FIG. 1M). Previous studies have shown that the intestinal microbiota remotely regulates cardiac function through metabolites (e.g., short-chain fatty acids,³⁸ trimethylamine N-oxide,³⁹ bile-acids¹¹) or by activating immune responses.^{29, 40} The glycopeptide vancomycin is clinically used for treating Clostridium Difficile infection.⁴¹⁻⁴³ It induces a distinct intestinal microbiota profile in humans and mice characterized by an increase in some Firmicutes (e.g., Lactobacillus sp.).³⁰⁻³² Thus, vancomycin-induced microbial reshaping prevents and mitigates pressure overload-induced myocardial hypertrophy and dysfunction.

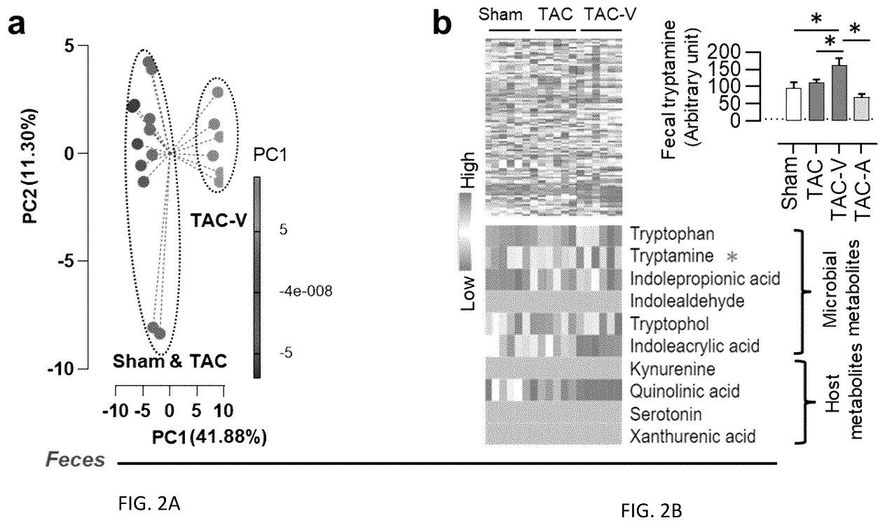

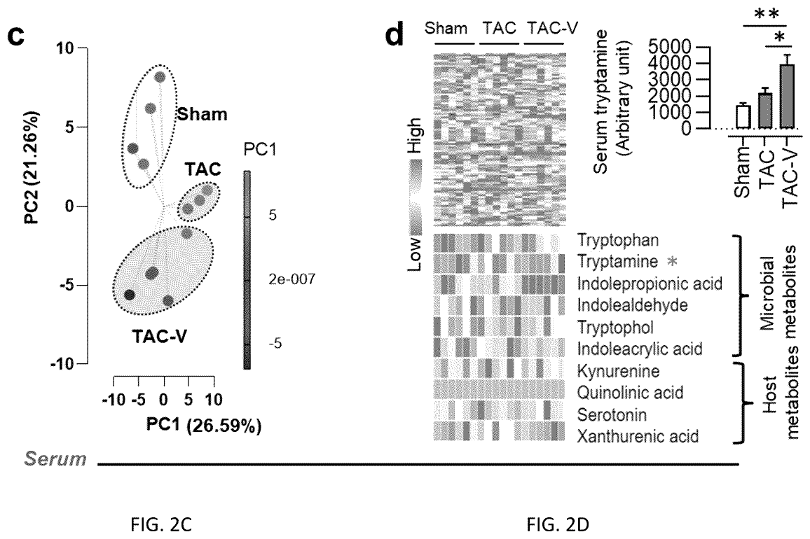

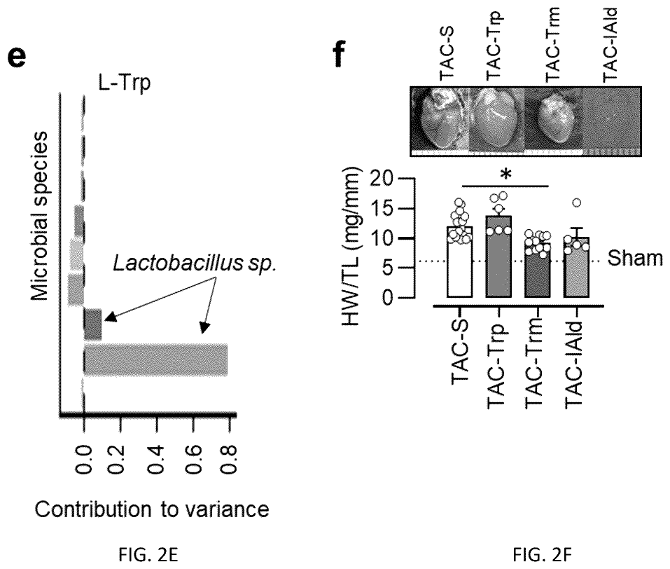

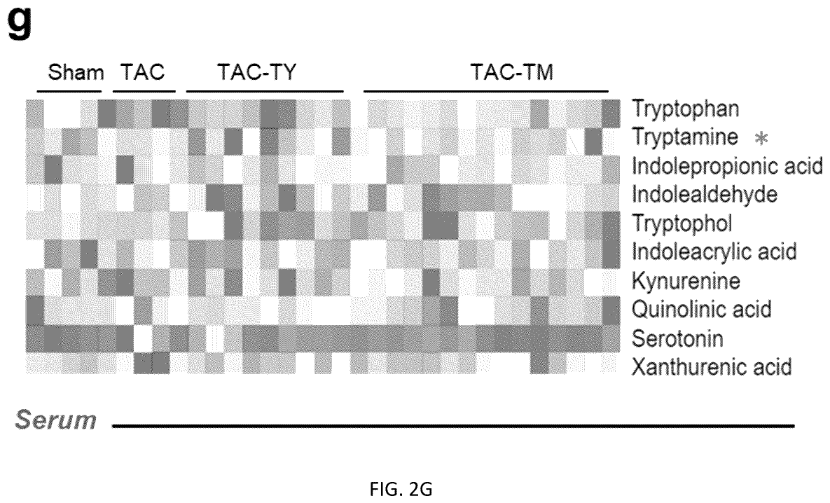

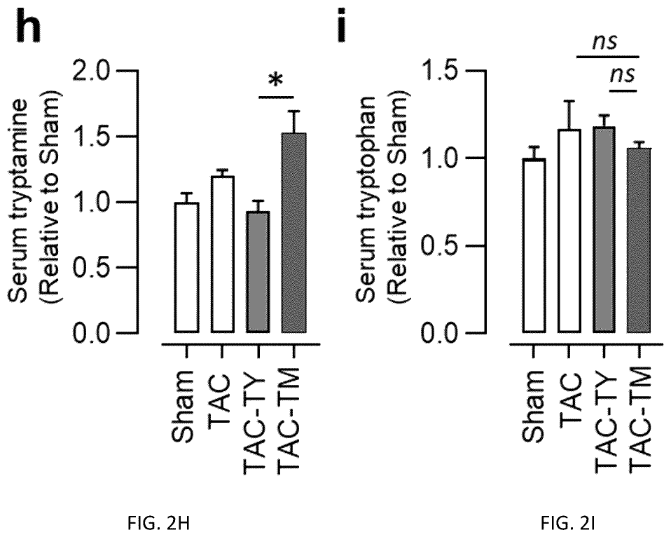

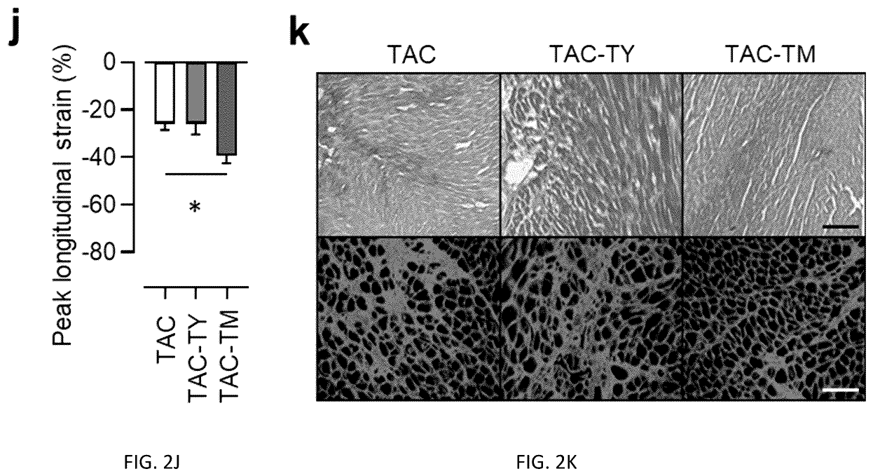

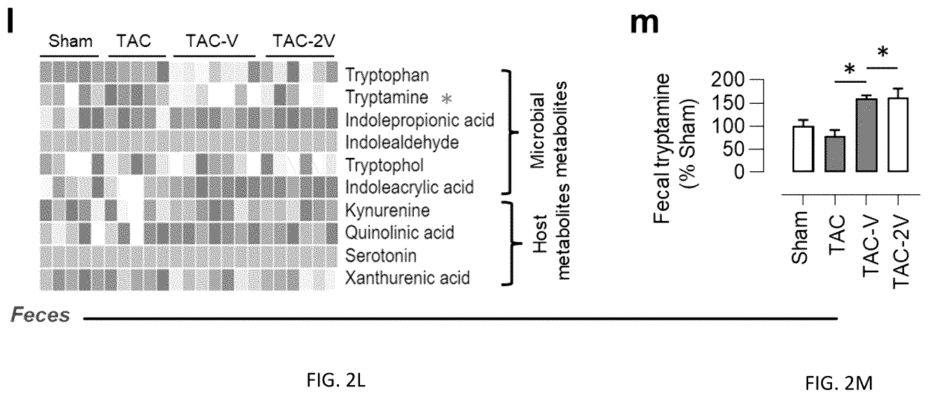

Vancomycin changes microbial indole metabolism in the gut. We used a multi-pronged approach to identify the secondary mediator of vancomycin-induced gut microbiota dysbiosis by analyzing the microbiota profile, metagenomics, and metabolomics. The cardiac impact of oral vancomycin, which has poor gastrointestinal absorption, suggests the presence of secondary mediators such as microbe-generated metabolites or immune cell mobilization from the intestinal wall in the circulation. We measured the effects of vancomycin on the fecal metabolites by gas chromatography/liquid chromatography-mass spectrometry and immune cell profile in the blood and peritoneal fluid by flow cytometry. The principal component analysis (PCA) and pairwise hierarchical clustering show that the metabolites in the feces of TAC-V mice are distinct from those of the Sham and TAC mice (FIGS. 2A and 2B). The co-inertia analysis of the fecal metabolites and fecal microbiota taxonomic profile was used to determine the global similarity, where a short distance between metabolome and microbiome implied a strong association. The microbiota and metabolomics of TAC-V were distinct from the sham and TAC group, primarily along the first axis. Next, a two-way orthogonal partial least square (O2PLS) analysis that models complex systemic variation was performed. It was found that the vancomycin changes specific microbes (e.g., Lactobacillus, Akkermansia, Parasutterella) and amino acids (e.g., valine, leucine, isoleucine, tryptophan). In addition, the metabolic function analysis showed an upregulation of the aminoacyl-tRNA biosynthesis and a higher level of indoles (e.g., tryptophan, tryptamine) in the feces. The effects of vancomycin on the serum metabolites were also measured. The PCA and pairwise hierarchical clustering show that the serum of TAC-V mice is distinct and has higher levels of tryptophan and tryptamine than that of the Sham and TAC mice (FIGS. 2C and 2D). An integrative metabolic model-based microbiome and metabolomics data analysis (MIMOSA2) was performed to identify if the variance of specific metabolites depends on the specific microbe.⁴⁴ Lactobacillus sp. strongly contributes to the catabolism of tryptophan (FIG. 2E). MetaboAnalyst's analysis of the feces and serum metabolic profile also identifies vancomycin's impact on tryptophan metabolism. Tryptamine concentrations increase over 100-fold in feces following colonization of germ-free mice with human gut microbiota,⁴⁵⁻⁴⁶ suggesting that gut bacteria produce it.

Microbial indole metabolite mediates the cardioprotective effects of microbial reshaping. To test if increased tryptophan or tryptamine or indole aldehyde in the gut mediates the cardioprotective effects, tryptophan (500 mg/kg/day, orally), or tryptamine (dose 12.5 mg/kg/day, orally), or indole aldehyde (dose 12.5 mg/kg/day, orally) was provided to the mice beginning one week before TAC surgery until five weeks post-TAC surgery. The tryptamine and indole aldehyde, but not tryptophan, inhibited TAC-induced myocardial hypertrophy and strain (FIG. 2F). No change in the serum tryptamine or tryptophan levels was noted in the tryptophan-treated mice (FIG. 2G-2I). One subset of mice that received tryptophan orally had lower tryptophan in the serum. However, the other subset of mice that received tryptophan orally had a higher tryptophan in the serum. Importantly, in both subsets, the serum tryptamine levels were relatively lower (FIG. 2G). These data suggest that tryptophan availability in the gut is insufficient to increase systemic tryptamine, and a second trigger of microbiota reshaping is needed to drive the tryptophan metabolism to tryptamine and to mediate cardioprotective effects. An improvement in the peak longitudinal strain, % EF, left ventricular weight, wall thickness, end-systolic/diastolic volume, fibrosis, and cross-sectional area of cardiomyocytes was found in TAC-TM (tryptamine-treated TAC) mice compared to the TAC-TY (tryptophan-treated TAC) mice (FIGS. 2J and 2K), all indicating lower susceptibility to the myocardial hypertrophy and dysfunction. Moreover, the hearts of TAC-TM mice had a decreased expression of load-response genes (nppa, nppb, and β-mhc) and collagen genes (Col1a1, Col1a2, and Col3a1), supporting that the mechanisms that contribute to fibrosis were not inhibited in the heart of TAC-TM mice. Since vancomycin reversed the early hypertrophic changes in the heart (FIGS. 1L and 1M), whether independent cohorts of mice also have an anticipated increase in the microbial generation of tryptamine in the gut was investigated. Notably, a similar increase in the fecal tryptamine levels in the gut of TAC-V and TAC-2V mice was found (FIGS. 2L and 2M). Furthermore, in the mice in which microbiota was reshaped using ampicillin did not protect the heart and was not associated with increased tryptamine levels in the gut (FIG. 2B, bar graph), supporting tryptamine as a mediator for the cardioprotective effects of vancomycin-induced microbial reshaping. The microbial generation of tryptamine would require tryptophan decarboxylase activity. Lactobacillus sp. catabolizes the tryptophan to tryptamine,⁴⁷⁻⁴⁹ but their genomic database does not show the presence of tryptophan decarboxylase (EC4.1.1.105). However, tyrosine decarboxylase, ornithine decarboxylase, and D-lactate dehydrogenase can catalyze tryptophan decarboxylation to tryptamine.⁴⁷⁻⁴⁹ The D-lactate dehydrogenase exhibits 40% homology with the clostridial tryptophan decarboxylase, producing tryptamine.⁴⁷ The intestinal abundance of Lactobacillus murinus also increases during calorie restriction, which improves cardiac health.³³ Other microbes with tryptophan decarboxylase activity can contribute to total tryptamine generation. These results show that vancomycin-induced microbial reshaping mediates cardioprotective effects via microbial indole metabolite tryptamine.

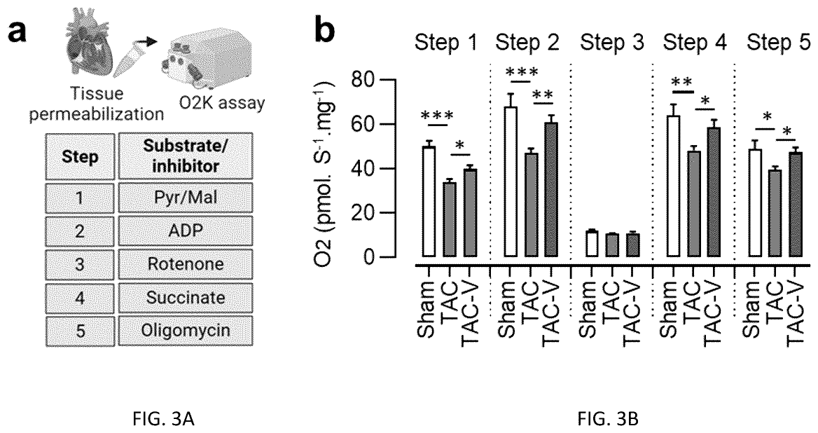

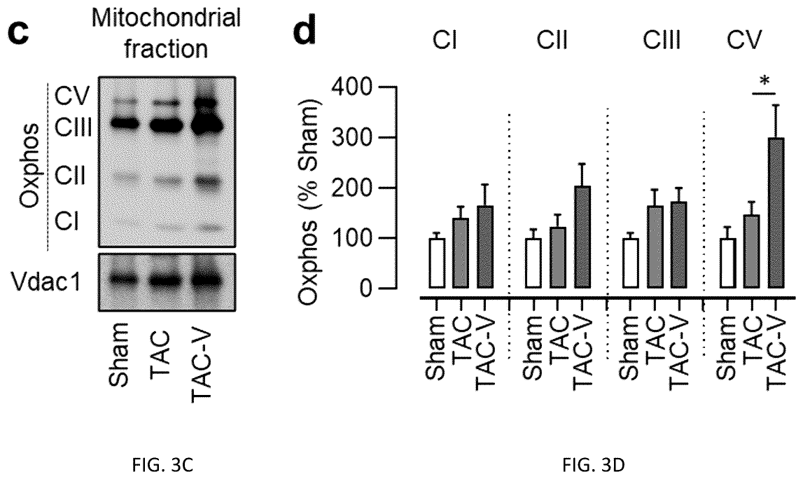

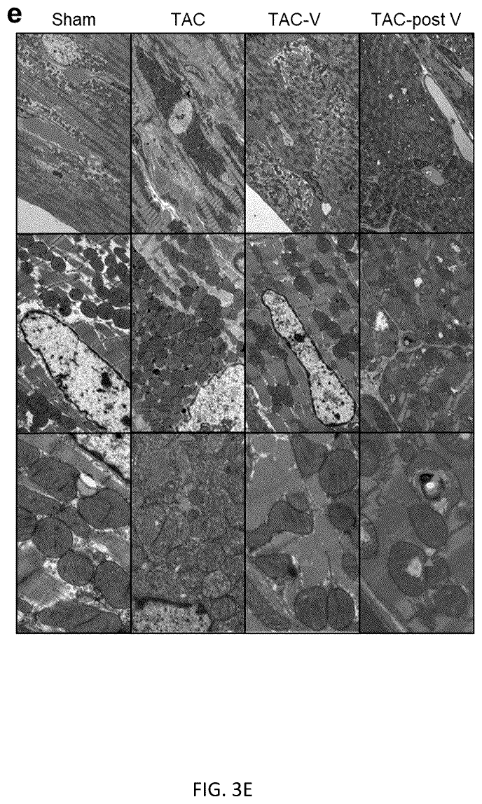

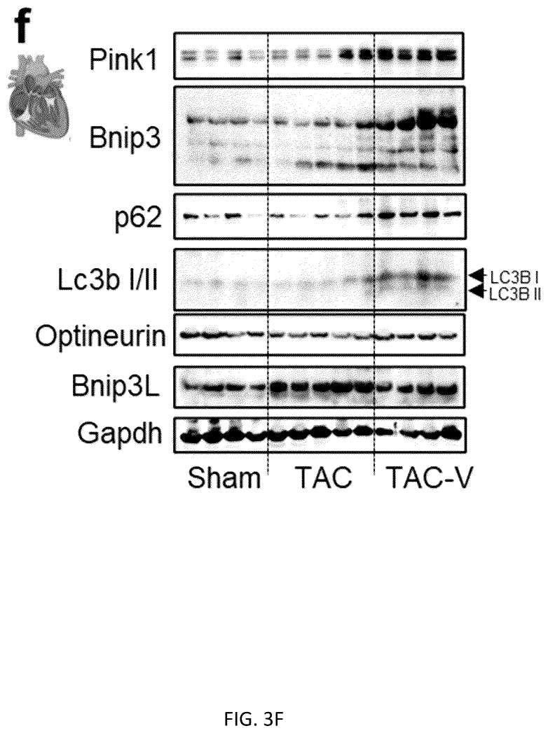

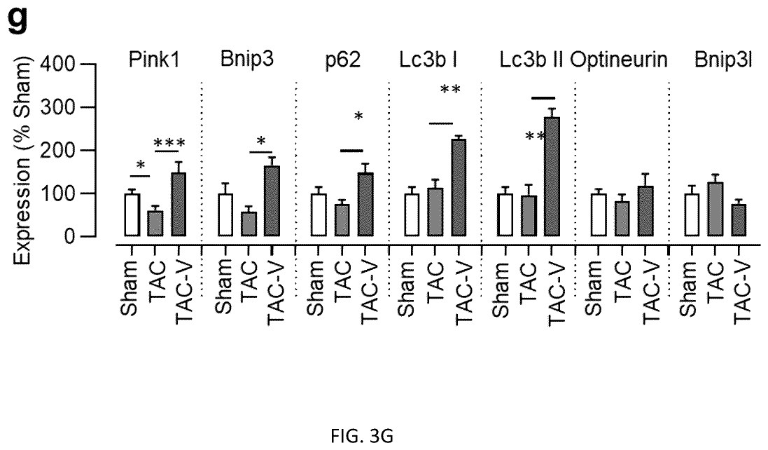

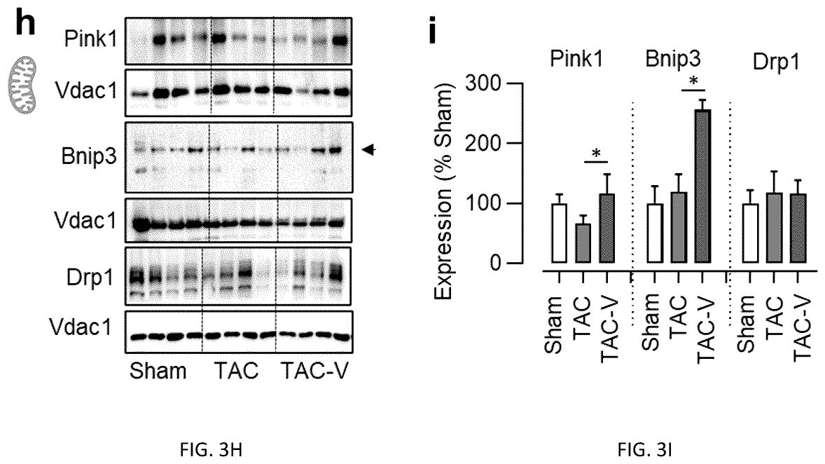

Vancomycin-induced microbial reshaping improves mitochondrial function in the heart. Oxygen consumption rate (OCR) of cardiac tissues isolated from the left ventricles was assessed (FIG. 3A). The pyruvate/malate-, ADP-induced OCR, signifying basal and stressed conditions, was restored in the hearts of TAC mice receiving vancomycin compared to TAC mice (FIG. 3B). Similarly, the succinate-induced OCR after rotenone-induced complex I inhibition, signifying the complex II contributions, were restored in the hearts of TAC mice receiving vancomycin compared to TAC mice (FIG. 3B). Higher expression of complex V (ATP synthase) was found in the mitochondrial fraction of the heart of TAC mice receiving vancomycin compared to TAC mice (FIGS. 3C and 3D). Cardiac ultrastructure by transmission electron microscopy showed an increase in mitochondrial abundance in the heart of TAC mice, which was not apparent in the TAC mice receiving vancomycin (FIG. 3E). In the mice in which vancomycin was used for two weeks after TAC surgery (TAC-post V), we observed several mitochondria at different stages of degradation, suggesting mitophagy (FIG. 3E). TAC initially activates mitophagy, which declines over time and contributes to mitochondrial dysfunction.⁵⁰ Pink1 and Bcl-2 interacting protein 3 (Bnip3) accumulation on the mitochondrial outer membrane promotes mitophagy.⁵¹ An increase in Pink1 and Bnip3 expression in the total heart lysate and the mitochondrial fraction of TAC-V mice hearts compared to TAC mice was noted (FIG. 3F-3I). The markers of ubiquitin-mediated mitophagy (e.g., p62, Lc3b, and ubiquitin) but not that of receptor-mediated mitophagy (e.g., Bnip31 and optineurin) were also higher in the TAC-V mice hearts (FIG. 3F-3G). The Dynamin-related peptide 1 (Drp 1) that regulates the mitochondrial fission remained unchanged in the mitochondrial fraction of the Sham, TAC, and TAC-V mice hearts (FIG. 3H-3I). Thus, microbial reshaping by vancomycin improves the mitochondrial.

### REFERENCES

1. Heianza, Y., Zheng, Y., Ma, W., Rimm, E. B., Albert, C. M., Hu, F. B., Rexrode, K. M., Manson, J. E. & Qi, L. Duration and life-stage of antibiotic use and risk of cardiovascular events in women. Eur Heart J 40, 3838-3845 (2019).(PMID: 31216010).
2. Luedde, M., Winkler, T., Heinsen, F. A., Ruhlemann, M. C., Spehlmann, M. E., Bajrovic, A., Lieb, W., Franke, A., Ott, S. J. & Frey, N. Heart failure is associated with depletion of core intestinal microbiota. ESC Heart Fail 4, 282-290 (2017).(PMID: 28772054).
3. Ianiro, G., Tilg, H. & Gasbarrini, A. Antibiotics as deep modulators of gut microbiota: between good and evil. Gut 65, 1906-1915 (2016).(PMID: 27531828).
4. Reijnders, D., Goossens, G. H., Hermes, G. D., Neis, E. P., van der Beek, C. M., Most, J., Holst, J. J., Lenaerts, K., Kootte, R. S., Nieuwdorp, M., Groen, A. K., Olde Damink, S. W., Boekschoten, M. V., Smidt, H., Zoetendal, E. G., Dejong, C. H. & Blaak, E. E. Effects of Gut Microbiota Manipulation by Antibiotics on Host Metabolism in Obese Humans: A Randomized Double-Blind Placebo-Controlled Trial. Cell Metab 24, 63-74 (2016).(PMID: 27411009).
5. Modi, S. R., Collins, J. J. & Relman, D. A. Antibiotics and the gut microbiota. J Clin Invest 124, 4212-4218 (2014).(PMID: 25271726).
6. Faber, F., Tran, L., Byndloss, M. X., Lopez, C. A., Velazquez, E. M., Kerrinnes, T., Nuccio, S. P., Wangdi, T., Fiehn, O., Tsolis, R. M. & Baumler, A. J. Host-mediated sugar oxidation promotes post-antibiotic pathogen expansion. Nature 534, 697-699 (2016).(PMID: 27309805).
7. Kelly, C. P., Pothoulakis, C. & LaMont, J. T. Clostridium difficile colitis. N Engl J Med 330, 257-262 (1994).(PMID: 8043060).
8. Jie, Z., Xia, H., Zhong, S. L., Feng, Q., Li, S., Liang, S., Zhong, H., Liu, Z., Gao, Y., Zhao, H., Zhang, D., Su, Z., Fang, Z., Lan, Z., Li, J., Xiao, L., Li, J., Li, R., Li, X., Li, F., Ren, H., Huang, Y., Peng, Y., Li, G., Wen, B., Dong, B., Chen, J. Y., Geng, Q. S., Zhang, Z. W., Yang, H., Wang, J., Wang, J., Zhang, X., Madsen, L., Brix, S., Ning, G., Xu, X., Liu, X., Hou, Y., Jia, H., He, K. & Kristiansen, K. The gut microbiome in atherosclerotic cardiovascular disease. Nat Commun 8, 845 (2017).(PMID: 29018189).
9. Marques, F. Z., Nelson, E., Chu, P. Y., Horlock, D., Fiedler, A., Ziemann, M., Tan, J. K., Kuruppu, S., Rajapakse, N. W., El-Osta, A., Mackay, C. R. & Kaye, D. M. High-Fiber Diet and Acetate Supplementation Change the Gut Microbiota and Prevent the Development of Hypertension and Heart Failure in Hypertensive Mice. Circulation 135, 964-977 (2017).(PMID: 27927713).
10. Organ, C. L., Otsuka, H., Bhushan, S., Wang, Z., Bradley, J., Trivedi, R., Polhemus, D. J., Tang, W. H., Wu, Y., Hazen, S. L. & Lefer, D. J. Choline Diet and Its Gut Microbe-Derived Metabolite, Trimethylamine N-Oxide, Exacerbate Pressure Overload-Induced Heart Failure. Circ Heart Fail 9, e002314 (2016).(PMID: 26699388).
11. Mayerhofer, C. C. K., Ueland, T., Broch, K., Vincent, R. P., Cross, G. F., Dahl, C. P., Aukrust, P., Gullestad, L., Hov, J. R. & Troseid, M. Increased Secondary/Primary Bile Acid Ratio in Chronic Heart Failure. J Card Fail (2017).(PMID: 28688889).
12. Liu, R., Hong, J., Xu, X., Feng, Q., Zhang, D., Gu, Y., Shi, J., Zhao, S., Liu, W., Wang, X., Xia, H., Liu, Z., Cui, B., Liang, P., Xi, L., Jin, J., Ying, X., Wang, X., Zhao, X., Li, W., Jia, H., Lan, Z., Li, F., Wang, R., Sun, Y., Yang, M., Shen, Y., Jie, Z., Li, J., Chen, X., Zhong, H., Xie, H., Zhang, Y., Gu, W., Deng, X., Shen, B., Xu, X., Yang, H., Xu, G., Bi, Y., Lai, S., Wang, J., Qi, L., Madsen, L., Wang, J., Ning, G., Kristiansen, K. & Wang, W. Gut microbiome and serum metabolome alterations in obesity and after weight-loss intervention. Nat Med 23, 859-868 (2017).(PMID: 28628112).
13. Palleja, A., Kashani, A., Allin, K. H., Nielsen, T., Zhang, C., Li, Y., Brach, T., Liang, S., Feng, Q., Jorgensen, N. B., Bojsen-Moller, K. N., Dirksen, C., Burgdorf, K. S., Holst, J. J., Madsbad, S., Wang, J., Pedersen, O., Hansen, T. & Arumugam, M. Roux-en-Y gastric bypass surgery of morbidly obese patients induces swift and persistent changes of the individual gut microbiota. Genome Med 8, 67 (2016).(PMID: 27306058).
14. Tremaroli, V., Karlsson, F., Werling, M., Stahlman, M., Kovatcheva-Datchary, P., Olbers, T., Fandriks, L., le Roux, C. W., Nielsen, J. & Backhed, F. Roux-en-Y Gastric Bypass and Vertical Banded Gastroplasty Induce Long-Term Changes on the Human Gut Microbiome Contributing to Fat Mass Regulation. Cell Metab 22, 228-238 (2015).(PMID: 26244932).
15. Zhang, H., DiBaise, J. K., Zuccolo, A., Kudrna, D., Braidotti, M., Yu, Y., Parameswaran, P., Crowell, M. D., Wing, R., Rittmann, B. E. & Krajmalnik-Brown, R. Human gut microbiota in obesity and after gastric bypass. Proc Natl Acad Sci USA 106, 2365-2370 (2009).(PMID: 19164560).
16. DiRienzo, D. B. Effect of probiotics on biomarkers of cardiovascular disease: implications for heart-healthy diets. Nutr Rev 72, 18-29 (2014).(PMID: 24330093).
17. Zhao, X., Duan, W., Sun, C., Li, Z., Liu, Y., Xiao, X., Wang, G., Gang, X. & Wang, G. Decreased Cardiovascular Risk after Roux-en-Y Gastric Bypass Surgery in Chinese Diabetic Patients with Obesity. J Diabetes Res 2017, U.S. Pat. No. 5,612,049 (2017).(PMID: 28744472).
18. Guo, Z., Liu, X. M., Zhang, Q. X., Shen, Z., Tian, F. W., Zhang, H., Sun, Z. H., Zhang, H. P. & Chen, W. Influence of consumption of probiotics on the plasma lipid profile: a meta-analysis of randomised controlled trials. Nutr Metab Cardiovasc Dis 21, 844-850 (2011).(PMID: 21930366).
19. Hendijani, F. & Akbari, V. Probiotic supplementation for management of cardiovascular risk factors in adults with type II diabetes: A systematic review and meta-analysis. Clin Nutr 37, 532-541 (2018).(PMID: 28318686).
20. Sharma, S., Kurpad, A. V. & Puri, S. Potential of probiotics in hypercholesterolemia: A meta-analysis. Indian J Public Health 60, 280-286 (2016).(PMID: 27976649).
21. Shimizu, M., Hashiguchi, M., Shiga, T., Tamura, H. O. & Mochizuki, M. Meta-Analysis: Effects of Probiotic Supplementation on Lipid Profiles in Normal to Mildly Hypercholesterolemic Individuals. PLoS One 10, e0139795 (2015).(PMID: 26473340).
22. Wu, Y., Zhang, Q., Ren, Y. & Ruan, Z. Effect of probiotic Lactobacillus on lipid profile: A systematic review and meta-analysis of randomized, controlled trials. PLoS One 12, e0178868 (2017).(PMID: 28594860).
23. Zheng, H. J., Guo, J., Wang, Q., Wang, L., Wang, Y., Zhang, F., Huang, W. J., Zhang, W., Liu, W. J. & Wang, Y. Probiotics, prebiotics, and synbiotics for the improvement of metabolic profiles in patients with chronic kidney disease: A systematic review and meta-analysis of randomized controlled trials. Crit Rev Food Sci Nutr 61, 577-598 (2021).(PMID: 32329633).
24. Vest, A. R., Heneghan, H. M., Agarwal, S., Schauer, P. R. & Young, J. B. Bariatric surgery and cardiovascular outcomes: a systematic review. Heart 98, 1763-1777 (2012).(PMID: 23077152).
25. Zhou, X., Yu, J., Li, L., Gloy, V. L., Nordmann, A., Tiboni, M., Li, Y. & Sun, X. Effects of Bariatric Surgery on Mortality, Cardiovascular Events, and Cancer Outcomes in Obese Patients: Systematic Review and Meta-analysis. Obes Surg 26, 2590-2601 (2016).(PMID: 26992897).
26. Chaudhry, U. I., Kanji, A., Sai-Sudhakar, C. B., Higgins, R. S. & Needleman, B. J. Laparoscopic sleeve gastrectomy in morbidly obese patients with end-stage heart failure and left ventricular assist device: medium-term results. Surg Obes Relat Dis 11, 88-93 (2015).(PMID: 25127439).
27. Ramani, G. V., McCloskey, C., Ramanathan, R. C. & Mathier, M. A. Safety and efficacy of bariatric surgery in morbidly obese patients with severe systolic heart failure. Clin Cardiol 31, 516-520 (2008).(PMID: 19006115).
28. Mistry, P., Reitz, C. J., Khatua, T. N., Rasouli, M., Oliphant, K., Young, M. E., Allen-Vercoe, E. & Martino, T. A. Circadian influence on the microbiome improves heart failure outcomes. J Mol Cell Cardiol 149, 54-72 (2020).(PMID: 32961201).
29. Gil-Cruz, C., Perez-Shibayama, C., De Martin, A., Ronchi, F., van der Borght, K., Niederer, R., Onder, L., Lutge, M., Novkovic, M., Nindl, V., Ramos, G., Arnoldini, M., Slack, E. M. C., Boivin-Jahns, V., Jahns, R., Wyss, M., Mooser, C., Lambrecht, B. N., Maeder, M. T., Rickli, H., Flatz, L., Eriksson, U., Geuking, M. B., McCoy, K. D. & Ludewig, B. Microbiota-derived peptide mimics drive lethal inflammatory cardiomyopathy. Science 366, 881-886 (2019).(PMID: 31727837).
30. Kim, A. H., Lee, Y., Kim, E., Ji, S. C., Chung, J. Y. & Cho, J. Y. Assessment of Oral Vancomycin-Induced Alterations in Gut Bacterial Microbiota and Metabolome of Healthy Men. Front Cell Infect Microbiol 11, 629438 (2021).(PMID: 34123865).
31. Ray, P., Pandey, U. & Aich, P. Comparative analysis of beneficial effects of vancomycin treatment on Th1- and Th2-biased mice and the role of gut microbiota. J Appl Microbiol 130, 1337-1356 (2021).(PMID: 32955795).
32. Isaac, S., Scher, J. U., Djukovic, A., Jimenez, N., Littman, D. R., Abramson, S. B., Pamer, E. G. & Ubeda, C. Short- and long-term effects of oral vancomycin on the human intestinal microbiota. J Antimicrob Chemother 72, 128-136 (2017).(PMID: 27707993).
33. Pan, F., Zhang, L., Li, M., Hu, Y., Zeng, B., Yuan, H., Zhao, L. & Zhang, C. Predominant gut Lactobacillus murinus strain mediates anti-inflammaging effects in calorie-restricted mice. Microbiome 6, 54 (2018).(PMID: 29562943).
34. Hoffman, M., Kyriazis, I. D., Lucchese, A. M., de Lucia, C., Piedepalumbo, M., Bauer, M., Schulze, P. C., Bonios, M. J., Koch, W. J. & Drosatos, K. Myocardial Strain and Cardiac Output are Preferable Measurements for Cardiac Dysfunction and Can Predict Mortality in Septic Mice. J Am Heart Assoc 8, e012260 (2019).(PMID: 31112430).
35. Mangalam, A., Shahi, S. K., Luckey, D., Karau, M., Marietta, E., Luo, N., Choung, R. S., Ju, J., Sompallae, R., Gibson-Corley, K., Patel, R., Rodriguez, M., David, C., Taneja, V. & Murray, J. Human Gut-Derived Commensal Bacteria Suppress CNS Inflammatory and Demyelinating Disease. Cell Rep 20, 1269-1277 (2017).(PMID: 28793252).
36. Gaddam, R. R., Jacobsen, V. P., Kim, Y. R., Gabani, M., Jacobs, J. S., Dhuri, K., Kumar, S., Kassan, M., Li, Q., Bahal, R., Roghair, R., Irani, K. & Vikram, A. Microbiota-governed microRNA-204 impairs endothelial function and blood pressure decline during inactivity in db/db mice. Sci Rep 10, 10065 (2020).(PMID: 32572127).
37. Souders, C. A., Borg, T. K., Banerjee, I. & Baudino, T. A. Pressure overload induces early morphological changes in the heart. Am J Pathol 181, 1226-1235 (2012).(PMID: 22954422).
38. Kaye, D. M., Shihata, W. A., Jama, H. A., Tsyganov, K., Ziemann, M., Kiriazis, H., Horlock, D., Vijay, A., Giam, B., Vinh, A., Johnson, C., Fiedler, A., Donner, D., Snelson, M., Coughlan, M. T., Phillips, S., Du, X. J., El-Osta, A., Drummond, G., Lambert, G. W., Spector, T. D., Valdes, A. M., Mackay, C. R. & Marques, F. Z. Deficiency of Prebiotic Fiber and Insufficient Signaling Through Gut Metabolite-Sensing Receptors Leads to Cardiovascular Disease. Circulation 141, 1393-1403 (2020).(PMID: 32093510).
39. Tang, W. H., Wang, Z., Fan, Y., Levison, B., Hazen, J. E., Donahue, L. M., Wu, Y. & Hazen, S. L. Prognostic value of elevated levels of intestinal microbe-generated metabolite trimethylamine-N-oxide in patients with heart failure: refining the gut hypothesis. J Am Coll Cardiol 64, 1908-1914 (2014).(PMID: 25444145).
40. Tang, T. W. H., Chen, H. C., Chen, C. Y., Yen, C. Y. T., Lin, C. J., Prajnamitra, R. P., Chen, L. L., Ruan, S. C., Lin, J. H., Lin, P. J., Lu, H. H., Kuo, C. W., Chang, C. M., Hall, A. D., Vivas, E. I., Shui, J. W., Chen, P., Hacker, T. A., Rey, F. E., Kamp, T. J. & Hsieh, P. C. H. Loss of Gut Microbiota Alters Immune System Composition and Cripples Postinfarction Cardiac Repair. Circulation 139, 647-659 (2019).(PMID: 30586712).
41. Matzke, G. R., Zhanel, G. G. & Guay, D. R. Clinical pharmacokinetics of vancomycin. Clin Pharmacokinet 11, 257-282 (1986).(PMID: 3530582).
42. Gonzales, M., Pepin, J., Frost, E. H., Carrier, J. C., Sirard, S., Fortier, L. C. & Valiquette, L. Faecal pharmacokinetics of orally administered vancomycin in patients with suspected Clostridium difficile infection. BMC Infect Dis 10, 363 (2010).(PMID: 21192802).
43. Rao, S., Kupfer, Y., Pagala, M., Chapnick, E. & Tessler, S. Systemic absorption of oral vancomycin in patients with Clostridium difficile infection. Scand J Infect Dis 43, 386-388 (2011).(PMID: 21198337).
44. Noecker, C., Eng, A., Muller, E. & Borenstein, E. MIMOSA2: A metabolic network-based tool for inferring mechanism-supported relationships in microbiome-metabolome data. Bioinformatics (2022).(PMID: 34999748).
45. Bhattarai, Y., Williams, B. B., Battaglioli, E. J., Whitaker, W. R., Till, L., Grover, M., Linden, D. R., Akiba, Y., Kandimalla, K. K., Zachos, N.C., Kaunitz, J. D., Sonnenburg, J. L., Fischbach, M. A., Farrugia, G. & Kashyap, P. C. Gut Microbiota-Produced Tryptamine Activates an Epithelial G-Protein-Coupled Receptor to Increase Colonic Secretion. Cell Host Microbe 23, 775-785 e775 (2018).(PMID: 29902441).
46. Marcobal, A., Kashyap, P. C., Nelson, T. A., Aronov, P. A., Donia, M. S., Spormann, A., Fischbach, M. A. & Sonnenburg, J. L. A metabolomic view of how the human gut microbiota impacts the host metabolome using humanized and gnotobiotic mice. ISME J 7, 1933-1943 (2013).(PMID: 23739052).
47. Montgomery, T. L., Eckstrom, K., Lile, K. H., Caldwell, S., Heney, E. R., Lahue, K. G., D′Alessandro, A., Wargo, M. J. & Krementsov, D. N. Lactobacillus reuteri tryptophan metabolism promotes host susceptibility to CNS autoimmunity. Microbiome 10, 198 (2022).(PMID: 36419205).
48. van Kessel, S. P., Frye, A. K., El-Gendy, A. O., Castejon, M., Keshavarzian, A., van Dijk, G. & El Aidy, S. Gut bacterial tyrosine decarboxylases restrict levels of levodopa in the treatment of Parkinson's disease. Nat Commun 10, 310 (2019).(PMID: 30659181).
49. Zhao, X., Stein, K. R., Chen, V., Griffin, M. E., Lairson, L. L. & Hang, H. C. Chemoproteomics reveals microbiota-derived aromatic monoamine agonists for GPRC5A. Nat Chem Biol (2023).(PMID: 37248411).
50. Shirakabe, A., Zhai, P., Ikeda, Y., Saito, T., Maejima, Y., Hsu, C. P., Nomura, M., Egashira, K., Levine, B. & Sadoshima, J. Drpl-Dependent Mitochondrial Autophagy Plays a Protective Role Against Pressure Overload-Induced Mitochondrial Dysfunction and Heart Failure. Circulation 133, 1249-1263 (2016).(PMID: 26915633).
51. Zhang, T., Xue, L., Li, L., Tang, C., Wan, Z., Wang, R., Tan, J., Tan, Y., Han, H., Tian, R., Billiar, T. R., Tao, W. A. & Zhang, Z. BNIP3 Protein Suppresses PINK1 Kinase Proteolytic Cleavage to Promote Mitophagy. J Biol Chem 291, 21616-21629 (2016).(PMID: 27528605).

We claim:

1. A method for treating and/or preventing a cardiac disorder, comprising administering one or more indole metabolite to a subject in need thereof.

2. The method of claim 1, wherein the one or more indole metabolite is selected from the group consisting of: tryptophan; tryptamine; indole propionic acid; indole aldehyde; indole acrylic acid; tryptophol; kynurenine; quinolinic acid; and xanthurenic acid.

3. The method of claim 2, wherein the one or more indole metabolite comprises tryptamine or indole aldehyde.

4. The method of claim 1, wherein the one or more indole metabolite is administered to the subject in an amount of about 10 mg/kg/day to about 500 mg/kg/day, or about 25 mg/kg/day to about 250 mg/kg/day.

5. The method of claim 1, further comprising administering one or more glycopeptide antibiotic to the subject in need thereof, prior to the administering one or more indole metabolites.

6. The method of claim 5, wherein the one or more glycopeptide antibiotic is selected from the group consisting of: vancomycin; teicoplanin; ramoplanin; oritavancin; dalbavancin; and telavancin.

7. The method of claim 6, wherein the glycopeptide antibiotic comprises vancomycin.

8. The method of claim 5, wherein the glycopeptide antibiotic is administered to the subject in an amount of about 10 mg/kg/day to about 2000 mg/kg/day.

9. The method of claim 5, wherein the glycopeptide antibiotic is administered orally.

10. The method of claim 1, wherein the subject in need thereof exhibits one or more risk factors for heart failure comprising hypertension, obesity, and type 2 diabetes.

11. The method of claim 5, wherein administering the glycopeptide antibiotic increases the abundance of Firmicutes in the gut of the subject, relative to the level of Firmicutes in the gut of the subject prior to the administering the glycopeptide antibiotic.

12. The method of claim 5, wherein administering the glycopeptide antibiotic increases the abundance of Lactobacillus sp. in the gut of the subject, relative to the level of Lactobacillus sp. in the gut of the subject prior to the administering the glycopeptide antibiotic.

13. The method of claim 5, wherein the administering the glycopeptide antibiotic decreases the abundance of Bacteroidetes, and increases the abundance of Firmicutes and/or Proteobacteria in the gut of the subject, relative to the level of Bacteroidetes, Firmicutes, and Proteobacteria in the gut of the subject prior to the administering the glycopeptide antibiotic.

14. The method of claim 1, wherein the one or more indole metabolite is present in a comestible product, a composition, or a nutritional supplement, or combinations thereof.

15. The method of claim 1, wherein the subject in need thereof, has, or is at risk of, one or more cardiac disorders selected from the group consisting of: coronary artery disease; cardiomyopathy; heart valve disease; aortic stenosis; and pericardial disease.

16. A kit for treating and/or preventing a cardiac disorder, comprising one or more indole metabolite and one or more glycopeptide antibiotic.

17. The kit of claim 16, wherein the one or more indole metabolite is selected from the group consisting of: tryptophan; tryptamine; indole propionic acid; indole aldehyde; indole acrylic acid; tryptophol; kynurenine; quinolinic acid; and xanthurenic acid.

18. The kit of claim 17, wherein the one or more indole metabolites comprises tryptamine or indole aldehyde.

19. The kit of claim 16, wherein the one or more glycopeptide antibiotic is selected from the group consisting of: vancomycin; teicoplanin; ramoplanin; oritavancin; dalbavancin; and telavancin.

20. The kit of claim 19, wherein the glycopeptide antibiotic comprises vancomycin.
